(19) United States
(12) Patent Application Publication
(10) Pub. No.: US 2026/0000285 A1
(43) Pub. Date: Jan. 1, 2026

(54) Speculum Device and Method of Use

(71) Applicant: UNIVERSITY OF KANSAS, Lawrence, KS (US)

(72) Inventor: Jennifer VILLWOCK, Shawnee, KS (US)

(21) Appl. No.: 19/247,145

(22) Filed: Jun. 24, 2025

Related U.S. Application Data
(60) Provisional application No. 63/664,369, filed on Jun. 26, 2024.

(51) Int. Cl. A61B 1/32

(52) U.S. Cl. CPC A61B 1/32; A61B 2560/04; A61B 2560/06

(57) ABSTRACT

A speculum may include an insertion portion coupled to a handle, the insertion portion comprising a non-expandable proximal shaft and an expandable distal region, the distal region having a distal region length l that is less than 50% of a full-length L of the insertion portion. The speculum further includes a deployment mechanism operably coupled to the distal region, wherein the proximal shaft comprises a first lumen configured for the passage of an endoscope and a second lumen configured for the passage of an instrument, wherein the deployment mechanism controls the movement of the expandable distal region between an open configuration and a closed configuration. In the closed configuration, a maximum width w of the distal region approximates a diameter D of the proximal shaft.

### CROSS-REFERENCE TO RELATED APPLICATIONS

This application claims priority to, and the benefit of, U.S. Provisional Patent Application No. 63/664,369, filed Jun. 26, 2024, the entire contents of which are incorporated herein by reference.

### BACKGROUND

The present disclosure relates generally to a speculum and its use. A speculum is a medical instrument used to widen a cavity in the body to facilitate examination and treatment. Speculums are most commonly associated with gynecological exams and procedures (e.g., pap smears, cervical biopsies, and the insertion of intrauterine devices), but they may be used in various medical fields.

The use of vaginal speculums has historically relied on direct line-of-sight, requiring a wide opening at the vaginal introitus. This method of use does not prioritize patient privacy and can be painful and/or uncomfortable. Thus, the structure, function, and materials used in existing speculums rarely create a comfortable experience for patients. Therefore, a need exists for improved speculum devices and methods of use.

### SUMMARY

The disclosed speculum device improves patient comfort and privacy. In some implementations of the present disclosure, the speculum device incorporates endoscopic access via an embedded port or lumen in the speculum. The endoscope enables photo documentation of cervical/vaginal findings, which is currently not part of routine practice. This process allows for the comparison of exams over time, improved patient education, creation of digital health applications from the data such as predictive algorithms based on the extent of cervical dilation to delivery of a baby, likelihood of cervical cancer and need for biopsy, and other long-term patient advantages.

Furthermore, the disclosed device may be used for patient home-monitoring with the option to relay imaging for telehealth-style applications. The speculum includes an angled shape with a handle, enabling patients to perform investigative procedures on their own.

One implementation of the present disclosure is a speculum for improving visualization of an internal anatomical structure. The speculum includes an insertion portion coupled to a handle, the insertion portion including a non-expandable proximal shaft and an expandable distal region. The distal region has a distal region length l that is less than 50% of a full length L of the insertion portion. The speculum further includes a deployment mechanism operably coupled to distal region. The proximal shaft includes a first lumen configured for passage of an endoscope and a second lumen configured for passage of an instrument. The deployment mechanism controls movement of the expandable distal region between an open configuration and a closed configuration. In the closed configuration a maximum width w of the distal region approximates a diameter D of the proximal shaft.

In some implementations, the speculum further includes a rotatable handle.

In some implementations, the proximal shaft is generally cylindrical.

In some implementations, the diameter D of the proximal shaft varies by no more than 10% along a length of the proximal shaft.

In some implementations, a diameter of the first lumen is 3 millimeters or less.

In some implementations, a diameter of the second lumen is less than the diameter of the first lumen.

In some implementations, the speculum further includes a third lumen configured to be fluidically coupled to a contrast well for delivery of contrast agent.

In some implementations, a starting point of the distal region is defined by a change in material from the proximal shaft.

In some implementations, the distal region length l is less than 40% of a full length L of the insertion portion.

In some implementations, the distal region length l varies circumferentially.

In some implementations, in the closed configuration, the maximum width w of the distal region is no more than 125% of the diameter D of the proximal shaft.

In some implementations, in the open configuration, the distal region widens distally between a starting point and a distal edge region.

In some implementations, in the open configuration, the distal region widens distally such that the maximum width w of the distal region is at least 250% of the diameter D of the proximal shaft.

In some implementations, in the open configuration, the distal region widens in at least three circumferential directions.

In some implementations, the distal region includes at least three petals.

In some implementations, at least one of the at least three petals includes a rigid, elongated tine enclosed in a leaflet, the leaflet having a lower durometer than tine.

In some implementations, the distal region includes a funnel that widens in all circumferential directions.

In some implementations, in the closed configuration, the funnel takes a retracted configuration, wherein the funnel is at least partially retracted within the proximal shaft.

In some implementations, in the closed configuration, the funnel takes a furled configuration.

In some implementations, the deployment mechanism includes an activator.

In some implementations, the activator is positioned on the handle.

In some implementations, the deployment mechanism includes an additional lumen extending at least partially between the activator and the distal region.

In some implementations, the deployment mechanism includes a fluid that travels through the additional lumen to fill the distal region in the open configuration.

In some implementations, the deployment mechanism includes a threaded portion and an internal rod extending at least partially between the activator and the threaded portion.

According to another implementation of the present disclosure, a method of using a speculum is disclosed. The includes inserting an insertion portion of the speculum into a patient, the insertion portion including a non-expandable proximal shaft and an expandable distal region. The distal region is less than 50% the length of the insertion portion. The method further includes activating a deployment mechanism. The method further includes expanding the distal region of the insertion portion from a closed configuration to an open configuration, thereby moving an internal tissue of the patient. The method further includes inserting an endoscope through a first lumen of the insertion portion. The method further includes moving a lens of the endoscope in relation to an anatomical structure of the patient. The method further includes visualizing the anatomical structure via the endoscope.

In some implementations, the method further includes rotating a handle of the speculum.

In some implementations, activating a deployment mechanism includes moving an activator.

In some implementations, moving an activator initiates translation of an internal rod.

In some implementations, activating a deployment mechanism further includes rotating at least one component of the deployment mechanism relative to a threaded portion.

In some implementations, moving an activator initiates movement of a fluid into distal region.

In some implementations, expanding the distal region includes expanding less than 50% of the full length L of the insertion portion such that the distal region widens distally between a starting point and a distal edge region.

In some implementations, the method further includes widening the distal region such that the maximum width w of the distal region is at least 300% of the diameter D of the proximal shaft.

In some implementations, expanding the distal region of the insertion portion includes expanding the distal region in at least three circumferential directions.

In some implementations, expanding the distal region of the insertion portion includes opening a funnel.

In some implementations, expanding the distal region of the insertion portion includes expanding at least three petals.

In some implementations, the method further includes rotating the insertion portion in the open configuration, thereby altering a view of the anatomical structure.

In some implementations, the method further includes capturing a first image of the anatomical structure at a first point in time, capturing a second image of the anatomical structure at a second point in time, and comparing the second image to the first image.

In some implementations, the second point in time is at least a week after the first point in time.

In some implementations, the first image and the second image are captured by the patient.

In some implementations, at least one of the first image and the second image are captured without in-person supervision of a medical practitioner.

In some implementations, a medical practitioner compares the second image to the first image.

In some implementations, visualizing the anatomical structure via the endoscope includes: processing the first image and the second image to create a model of the anatomical structure.

In some implementations, the model of the anatomical structure is a three-dimensional model formed by the combination of the first image and the second image.

In some implementations, the three-dimensional model is formed by the combination of a plurality of images captured by the endoscope.

In some implementations, the first image is taken by the endoscope at a first position and the second image is taken by the endoscope at a second position that is angled with respect to the first position.

In some implementations, visualizing the anatomical structure via the endoscope includes: capturing, via one or more cameras of the endoscope, visual data of the anatomical structure; and compiling, via a controller in communication with the endoscope, the visual data of the anatomical structure into a visual model of the anatomical structure.

In some implementations, the method further includes displaying, via a user interface coupled to the controller, the visual model of the anatomical structure.

In some implementations, the method further includes continuously updating the three-dimensional model based on continuously collected visual data.

According to another implementation of the present disclosure, a system for visualizing an internal anatomical structure is disclosed. The system includes a speculum including an insertion portion coupled to a handle, the insertion portion including a non-expandable proximal shaft and an expandable distal region. The distal region has a distal region length l that is less than 50% of a full length L of the insertion portion. The speculum further includes a deployment mechanism operably coupled to distal region. The proximal shaft includes a first lumen configured for passage of an endoscope including at least one camera on a distal end thereof and a second lumen configured for passage of an instrument. The deployment mechanism controls movement of the expandable distal region between an open configuration and a closed configuration. In the closed configuration a maximum width w of the distal region approximates a diameter D of the proximal shaft. The system further includes a controller in communication with the endoscope. The controller includes a memory configured to receive and store visual data from the at least one camera of the endoscope. The controller further includes a processor configured to compile a plurality of images from the visual data to create a processed visual data set.

In some implementations, the system further includes a user interface configured to display the processed visual data set from the at least one camera of the endoscope.

In some implementations, the visual data captured by the endoscope includes a plurality of images taken from a plurality of angles.

In some implementations, the visual data captured by the endoscope includes a video captured by the at least one camera.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 shows a front isometric view of a speculum, according to one implementation.

FIG. 2 shows a back isometric view of the speculum of FIG. 1.

FIG. 3 shows a side view of a distal end of a speculum, according to one implementation.

FIG. 4 shows a side view of a distal end of a speculum, according to one implementation.

FIG. 5 shows a side view of a distal end of a speculum, according to one implementation.

FIG. 6 shows a cross-sectional view of the distal end of the speculum shown in FIG. 5.

FIG. 7 shows a diagram of a deployment mechanism on the distal end of a speculum, according to one implementation.

FIG. 8 shows a deployment mechanism and a handle of a speculum, according to one implementation.

FIG. 9 shows an isometric view of a speculum, according to one implementation.

FIG. 10 shows a distal end of the speculum of FIG. 9 in the closed configuration.

FIG. 11 shows a back isometric view of the speculum of FIG. 9 in the open configuration.

FIG. 12 shows a distal end of the speculum of FIG. 9 in the open configuration.

FIG. 13 shows a detailed view of a petal, according to one implementation.

FIG. 14 shows an example speculum in use in a patient, wherein the expandable distal region is in the open configuration, according to one implementation.

FIG. 15 shows an alternative deployment system in a speculum in the closed configuration, according to one implementation.

FIG. 16 shows the alternative deployment system of FIG. 15 in the open configuration.

FIG. 17 shows a flowchart of a method of using the disclosed device and/or system, according to one implementation.

FIG. 18 shows a diagram of a computing device and associated components, according to one implementation.

The device is explained in even greater detail in the following drawings. The drawings are merely exemplary and certain features may be used singularly or in combination with other features. The drawings are not necessarily drawn to scale.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a novel vaginal speculum is shown, according to various implementations. The disclosed speculum minimizes internal tissue displacement and the size of the opening at the vaginal introitus. In some implementations, the speculum features a stationary shaft, which includes a port through which a small visualization instrument such as a camera or endoscope can be introduced. Through the lumen of the shaft, additional instruments can also be passed so that procedures such as cervical or vaginal biopsy, culture, and smears can be performed. The distal end of the device may include 3-6 deployable tines with rounded edges to displace vaginal tissue adjacent to the cervix. The opening mechanism may rely on pneumatics, hydraulics, spring, screw, sliding, or other deployment mechanisms. The disclosed deployment mechanism deploys the tines distally while maintaining a narrow shaft diameter. In some implementations, once deployed, the tines remain deployed until the deployment mechanism is disengaged.

### EXAMPLE DEVICE #1

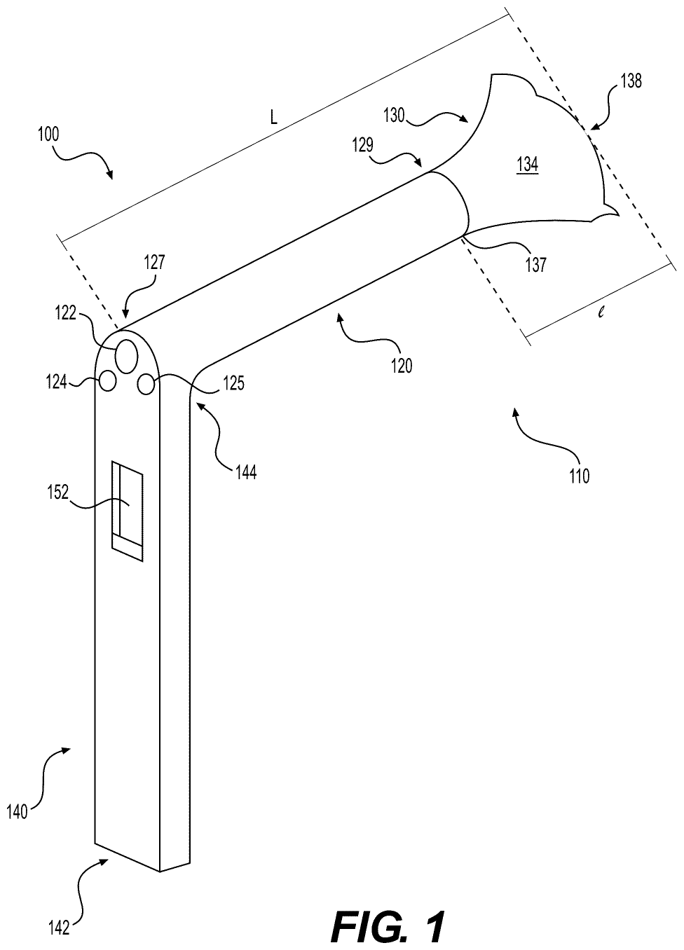

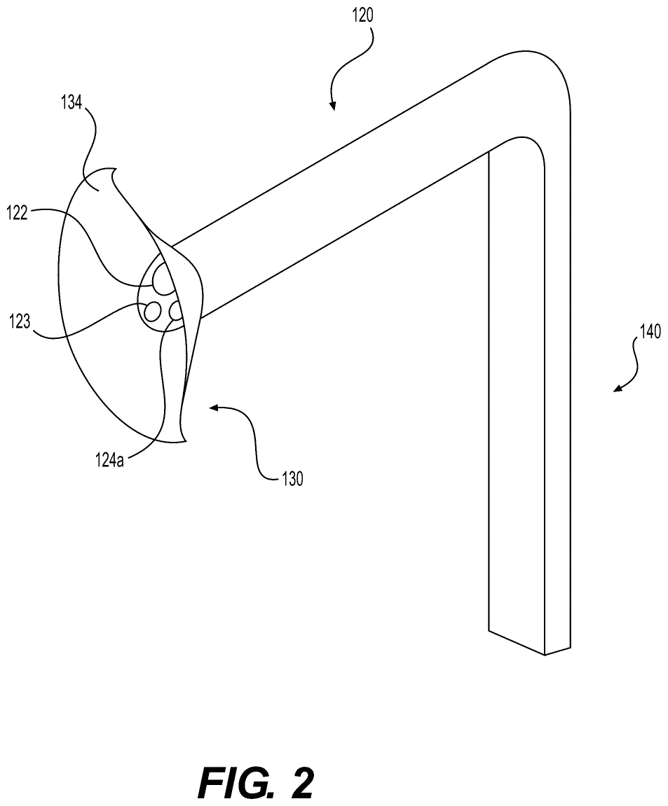

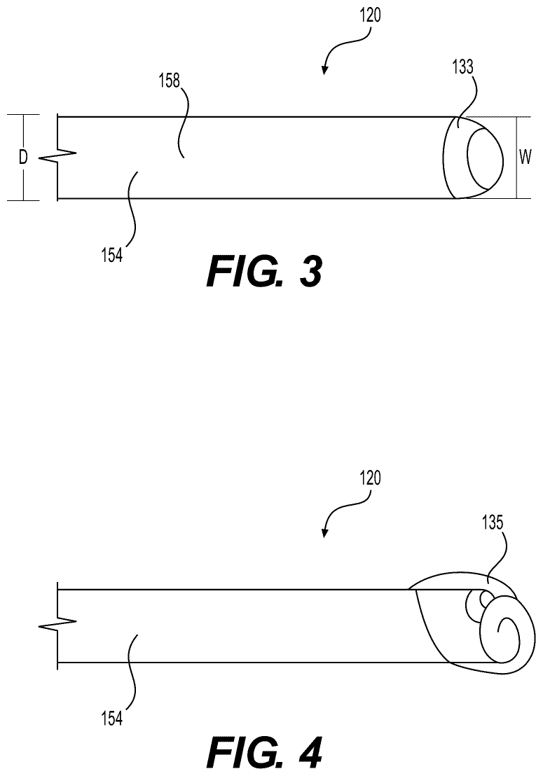

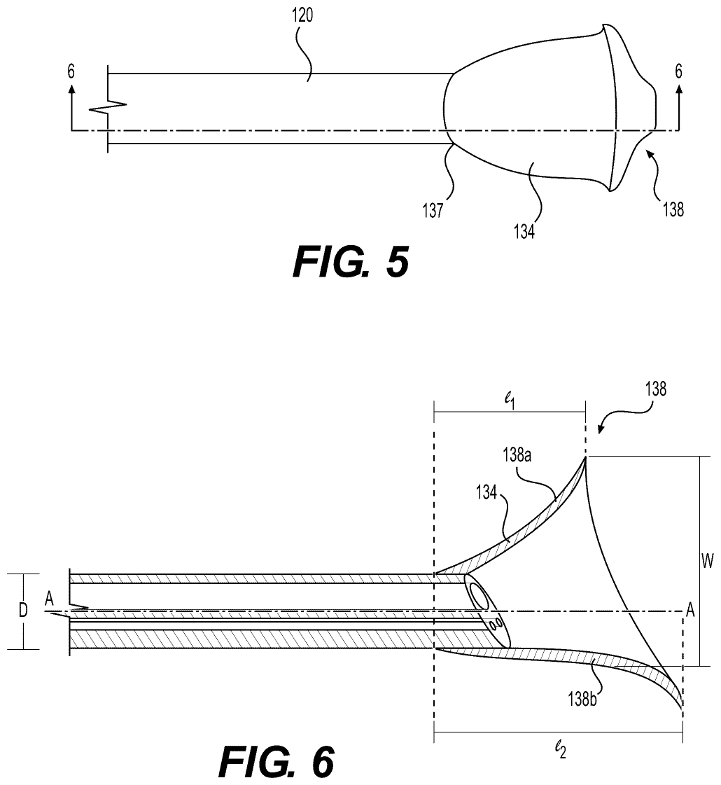

FIG. 1 shows a front isometric view of a speculum 100, according to one implementation. FIG. 2 shows a back isometric view of the speculum 100 of FIG. 1. FIGS. 3, 4, and 5 each show a side view of a distal end of the speculum 100 in various configurations. FIG. 6 shows a cross-sectional view of the distal end of the speculum shown in FIG. 5. The speculum 100 may be used, for example, for visualization of an internal anatomical structure of a user (e.g., visualization of the cervix by a user with or without the help of a healthcare professional).

The speculum 100 includes an insertion portion 110 coupled to a handle 140. The handle 140 extends from a first end 142 to a second end 144 opposite and spaced apart from the first end 142 of the handle 140. The handle 140 is rigidly formed with the insertion portion 110. However, in other implementations, the handle may be rotatable or bendable relative to the insertion portion.

The insertion portion 110 includes a non-expandable proximal shaft 120 and an expandable distal region 130. The proximal shaft 120 of the insertion portion 110 includes a proximal end 127 coupled to and integrally formed with the second end 144 of the handle 140. The proximal shaft 120 extends from the proximal end 127 to a distal end 129 opposite from the proximal end 127 of the proximal shaft 120.

The expandable distal region 130 is coupled to the distal end 129 of the proximal shaft 120. The expandable distal region 130 is defined between a starting point 137 coincident with the distal end 129 of the proximal shaft 120 and a distal edge region 138 opposite from the starting point 137. In some implementations, a starting point 137 of expandable distal region 130 can begin where the distal region 130 begins to widen outwardly relative to the non-expandable proximal shaft 120. In the implementation shown, the starting point 137 is defined by a change in material from the proximal shaft 120 to the expandable distal region 130 (e.g., from a rigid plastic to a flexible silicone material). However, in other implementations, the materials may change more gradually.

The handle 140 and the insertion portion 110 (including the proximal shaft 120) are generally longitudinal members that extend perpendicular to each other (e.g., forming a right-angle speculum 100). However, in other implementations, the handle and the insertion portion may form a different angle between each other (e.g., an angle between 0 and 180 degrees such as 30 degrees, 45 degrees, or 60 degrees). In other implementations, the handle and the insertion portion may be longitudinal members having more than one portion disposed at an angle with respect to another portion (e.g., a handle having a proximal and distal portion disposed at an angle with respect to each other). The overall geometry and structure of the speculums of this disclosure may be modified to fit a specific anatomical structure or medical function. Such variations and implementations are contemplated by this disclosure.

In some implementations, the handle 140 and the insertion portion 110 are rotatable with respect to one another. For example, the handle 140 may be coupled to the insertion portion 110 via a joint that enables rotation of either the handle 140 or the insertion portion 110 with respect to the other. In some implementations, the handle 140 can rotate about a longitudinal axis of the insertion portion 110. In some implementations, the insertion portion 110 can rotate about a longitudinal axis of the handle 140. In some implementations, either the handle 140 or the insertion portion 110 is rotatable within a plane parallel to the longitudinal axes of each of the handle 140 and the insertion portion.

A length l of the expandable distal region 130 is defined between the starting point 137 and the distal edge region 138 (e.g., when the expandable distal region 130 is in an open or deployed configuration). An overall length L of the insertion portion 110 is defined between (i) the proximal end 127 of the proximal shaft 120, and (ii) the distal edge region 138 of the expandable distal region 130. The length l of the expandable distal region 130 is less than 50% of the full length L of the insertion portion 110. In some implementations, the length l of the expandable distal region 130 is less than 40% of the full length L of the insertion portion 110 (e.g., 30%, 20%, 10%, 5%, or 1%). For example, the length L of the insertion portion 110 may be 15 cm while the length l of the expandable distal region 130 may be 3.5 cm. In some implementations, the length l of the expandable distal region 130 varies circumferentially (e.g., in the case of an asymmetrical funnel shape, such as in FIG. 6).

The proximal shaft 120 is generally cylindrical in shape, having a circular cross section. For example, in some implementations, the diameter of the proximal shaft 120 varies by no more than 10% along its length. In some implementations, the proximal shaft may include another shape easily insertable into an anatomical structure (e.g., having an elliptical or ovular cross section).

The proximal shaft 120 includes a first lumen 122, a second lumen 124, and a third lumen 125, each depicted in FIGS. 3-6. Each lumen includes an opening shown on an outer surface of the handle 140 axially aligned with the proximal shaft 120. While three lumens are included in the proximal shaft 120 of FIGS. 1-2, in other implementations, the proximal shaft may include more than three lumens or less than three lumens (e.g., a single lumen). However, the scope is not limited to the three lumens shown in FIG. 1. In some implementations, more or fewer than three lumens may be utilized. For example, in one implementation, a single lumen with a wide diameter may be utilized for the passage of multiple instruments.

The first lumen 122 is 7 mm in diameter. However, in other implementations, the first lumen is less than 7 mm in diameter (e.g., 5 mm or 3 mm). The second lumen 124 and the third lumen 125 are each smaller in diameter than the first lumen 122. The first lumen 122 is configured for the passage of an endoscope through the proximal shaft 120 and adjacent to the expandable distal region 130. The endoscope may be electrically coupled to a controller and configured to collect images of the anatomical structure when desired.

The second lumen 124 is configured for the passage of an instrument (e.g., a medical or surgical instrument). The third lumen 125 is configured for the delivery of a contrast agent, and it may be fluidically coupled to a contrast well or source. In other implementations, any one of the lumens described herein may be configured for a different function or delivery operation. In other implementations, one or more of the lumens may be coupled to a portion of the expandable distal region 130.

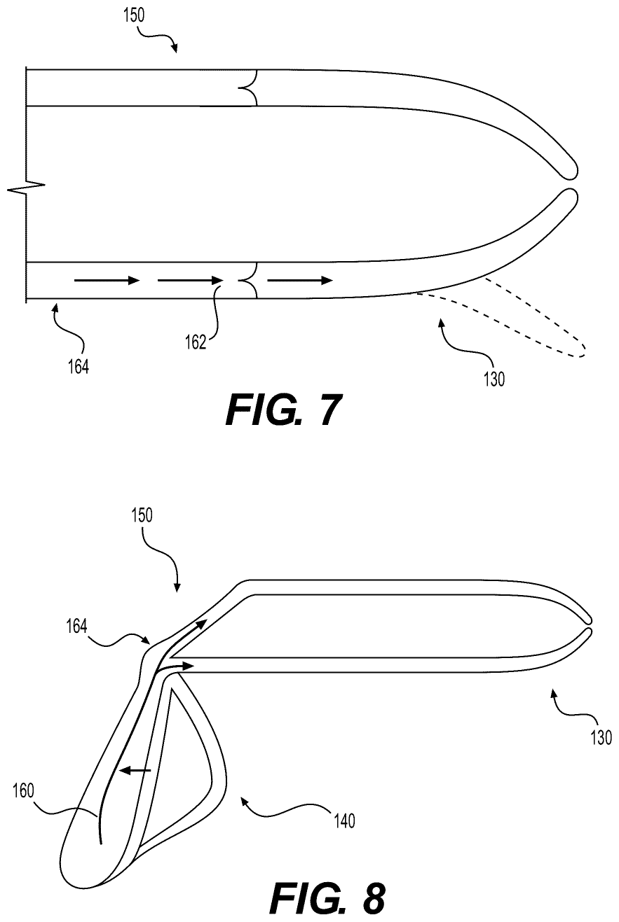

A deployment mechanism 150 is operably coupled to the expandable distal region 130. As shown in FIG. 7, the deployment mechanism 150 is disposed within the proximal shaft 120 and extends up to the expandable distal region 130. The deployment mechanism 150 may further extend up to and/or beyond the handle 140, as shown in FIG. 8, depending on the implementation of the deployment mechanism.

The deployment mechanism 150 controls the movement of the expandable distal region 130 between an open configuration (e.g., as shown in FIGS. 1, 2, 5, and 6), and a closed configuration (e.g., as shown in FIG. 3). The shadow lines on FIG. 7 further depict this movement between an open and a closed configuration. The deployment mechanism 150 provides for continuous control of the expandable distal region 130 between an open and closed configuration. The deployment mechanism 150 can move the expandable distal region 130 anywhere between the open and closed configuration (e.g., 50% open, 25% open, 25% closed, etc.).

In the closed configuration, the maximum width W of the distal region 130 approximates the diameter D of the proximal shaft 120 (e.g., as shown in FIGS. 3 and 4). For example, the maximum width W of the expandable distal region 130 in the closed configuration may be no more than 125% of the diameter D of the proximal shaft 120. In the open configuration, the maximum width W of the distal region 130 is greater than the diameter D of the proximal shaft 120 (e.g., as shown in FIG. 6). For example, the maximum width W of the expandable distal region 130 in the open configuration may be at least 250% of the diameter D of the proximal shaft 120.

FIG. 3 shows the expandable distal region 130 in a closed configuration, before activation. Once the deployment mechanism 150 begins to controllably activate the expandable distal region 130 to move towards the open configuration, the expandable distal region 130 expands radially and longitudinally outward from the proximal shaft 120, as shown in FIG. 4. The fully deployed open configuration of the expandable distal region 130 is shown in FIG. 5 and the cross-section of FIG. 6.

The fully deployed expandable distal region 130 widens distally between the starting point 137 and the distal edge region 138, forming a funnel or flower-like shape. As seen in FIG. 4, the expandable distal region 130 unfurls as it expands towards the open configuration, similar to a flower bud expanding. Thus, in the closed configuration, the expandable distal region 130 is furled or folded on itself in a compact structure. In some implementations, the expandable distal region 130 may partially retract into the proximal shaft 120 in the closed configuration.

The overall maximum width of the expandable distal region 130 occurs at or near the distal edge region 138, wherein the maximum width W is measured between opposing sides of the distal edge region 138. For example, FIG. 6 shows a first side 138a of the distal edge region 138 and a second side 138b of the distal edge region 138. The example shown in FIG. 6 includes an asymmetric expandable distal region 130 such that the first side 138a expands radially outward further than the second side 138b. However, the second side 138b expands axially further than the first side 138a, creating an asymmetry in the distal edge region 138. Such an arrangement may be used when, for example, the cervix of a user is facing posteriorly, and the longer “petal” or the second side 138b can push away the posterior vaginal tissue for better visualization.

The deployment mechanism 150 for moving the expandable distal region 130 between the open and closed configuration may include a variety of actuation methods. In a first example, the deployment mechanism 150 is a hydraulic or pneumatic deployment mechanism that delivers a pressurized fluid to the expandable distal region 130 to activate and expand the expandable distal region 130. For example, FIGS. 7 and 8 show a detail view of a pneumatic or hydraulic system for expanding opposing sides (e.g., petals) of the expandable distal region 130. FIGS. 6.5A and 6.5B are shown as a cross-section, similar to FIG. 6. Thus, while it appears that two sides or “petals” are provided, the expandable distal region 130 is a funnel-like structure extending circumferentially around the distal edge region 138. However, in other implementations, the expandable distal region includes discrete structures or “petals” each extendable from the starting point 137 adjacent to the proximal shaft 120 (e.g., three or more discrete petals).

As shown in FIGS. 7 and 8, a fluid source 160 may be coupled to the handle 140 of the speculum 100, either directly or via a conduit. The fluid source 160 is in fluid communication with a lumen 164 (e.g., any one of the lumens 122, 124, 125; or a separate lumen extending through the proximal shaft 120). The lumen 164 is in fluid communication with an inner channel 162 defined by the expandable distal region 130.

In the closed or relaxed configuration, the expandable distal region 130 is biased radially inward. The biased nature of the expandable distal region 130 may be a result of the material properties (e.g., shape memory material or discontinuous stiffness of the material) or the structure inherent in the expandable distal region 130. Once the deployment mechanism 150 urges pressurized fluid into the lumen 164 of the expandable distal region 130, the expandable distal region 130 begins to straighten out and expand radially outward opposite of the biased direction, as shown in FIG. 7. The fluid pressure thus causes the expandable distal region 130 to expand to the open or deployed configuration.

The fluid may be dispensed from the fluid source 160 and into the lumen 164 via a pump (e.g., a hand-squeeze pump on the handle 140, a controlled pump coupled to the handle 140, or a controlled pump separate from the handle 140). In some implementations, a button or trigger on the handle 140 is used to activate the deployment mechanism 150 (e.g., button 152 of FIG. 1). In some implementations, a syringe with a luer lock may be used to controllably deliver fluid into the expandable distal region 130.

In some implementations, one or more valves may be included in the expandable distal region 130 in proximity to the distal edge region 138 and/or the starting point 137 of the expandable distal region 130. The valves may be configured to maintain the expandable distal region 130 in the open/expanded configuration (e.g., so that a user need not continuously squeeze the handle 140 to keep the expandable distal region 130 open).

In use, a user (e.g., a patient or a healthcare professional) inserts the insertion portion 110 of the speculum 100 into the patient. The shape of the speculum 100 and the handle 140 allow for a patient to use the speculum 100 on their own if desired. The narrow and substantially straight insertion portion 110 facilitates the insertion of the speculum 100, in contrast to existing speculum devices which include large, tapered structures with a large proximal diameter.

Once the expandable distal region 130 of the speculum 100 is adjacent to a desired anatomical feature (e.g., a cervix of the patient), the user activates the deployment mechanism 150. The deployment may include activating a button, switch, slider, or other mechanical deployment mechanism coupled to the handle 140. The deployment mechanism 150 may then inject fluid into the expandable distal region 130 to expand the expandable distal region 130 from the closed configuration to the open configuration. In contrast to existing speculum devices which expand along a majority of the length of the speculum, the disclosed device expands only at the expandable distal region 130, improving patient comfort.

The open configuration of the expandable distal region 130 pushes the internal tissue of the patient away from the distal end 129 of the proximal shaft 120. This provides a clear pathway for endoscopes or other medical tools inserted through the insertion portion 110 (e.g., via the one or more lumens). The expanded anatomy allows for endoscopic viewing without the need for uncomfortable line-of-sight viewing.

Next, an endoscope is inserted through the first lumen 122 of the insertion portion 110. The endoscope includes, at least, a light and at least one camera/lens. The distal end of the endoscope with the camera is moved out of the proximal shaft 120 and near to an anatomical structure. The user can then visualize the anatomical structure (e.g., via a screen coupled to the endoscope). In some implementations, the insertion portion 110 may be rotated such that a different portion or viewpoint of the anatomical structure is visible.

In some implementations, the endoscope and attached controller are configured to capture images of the anatomical structure. For example, images may be taken at a first time and at a second time (e.g., hours, days, or weeks apart from each other). The images may be stored on a memory device of the speculum 100 or the associated controller for analysis by the patient or a medical professional.

### EXAMPLE DEVICE #2

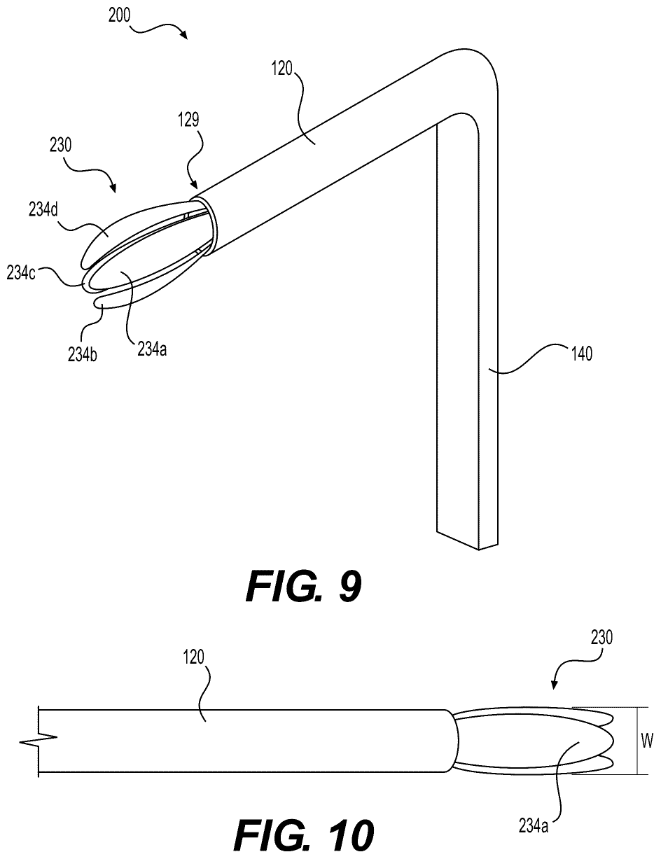

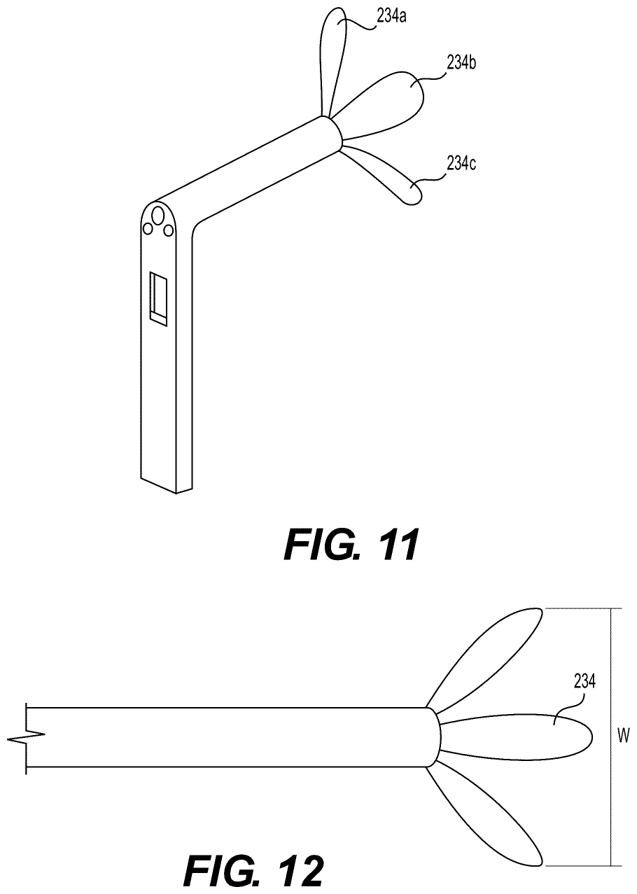

FIG. 9 shows a speculum 200 that is similar in structure and function as the speculum 100, except as described below. FIG. 10 shows a distal end of the speculum 200 in the closed configuration. FIG. 11 and FIG. 12 show the speculum 200 in the open configuration. The speculum 200 includes a handle 140 and a proximal shaft 120 similar to the speculum 100. However, the speculum 200 includes an expandable distal region 230 having discrete petals 234a, 234b, 234c, and 234d.

The expandable distal region 230, unlike the expandable distal region 130 of speculum 100, includes multiple, individual petals 234 which radially and longitudinally expand out from the distal end 129 of the proximal shaft 120. As shown in FIG. 9, four petals 234a, 234b, 234c, and 234d are included. However, in other implementations, any number of three or more petals may be included.

The petals 234a-234d may be individually actuated or collectively activated by a deployment mechanism 150. For example, each of the petals 234a-234d may be coupled to and in fluid communication with a lumen (or a manifold coupled to a lumen) for the delivery of pressurized fluid. The fluid expands the petals 234a-234d in a manner similar to the expandable distal region 130.

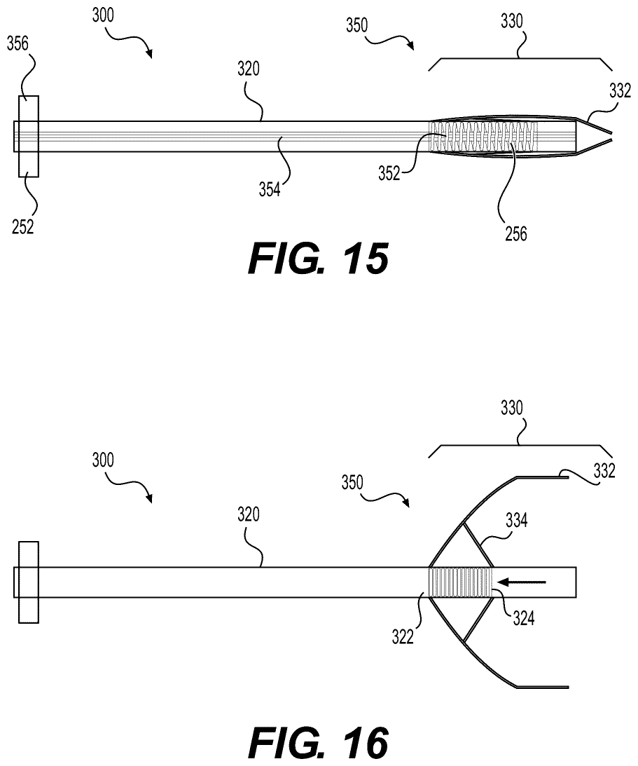

In other implementations, (e.g., in FIGS. 15-16) a shaft extends along the insertion portion 110 to the expandable distal region 230 to couple to a proximal end of each of the petals 234a-234d. The shaft is axially extendable to move the expandable distal region 230 from a closed configuration (e.g., wherein the petals are retained by the proximal shaft 120), to the open configuration (e.g., wherein the petals extend axially away from the proximal shaft 120 and radially expand away from each other).

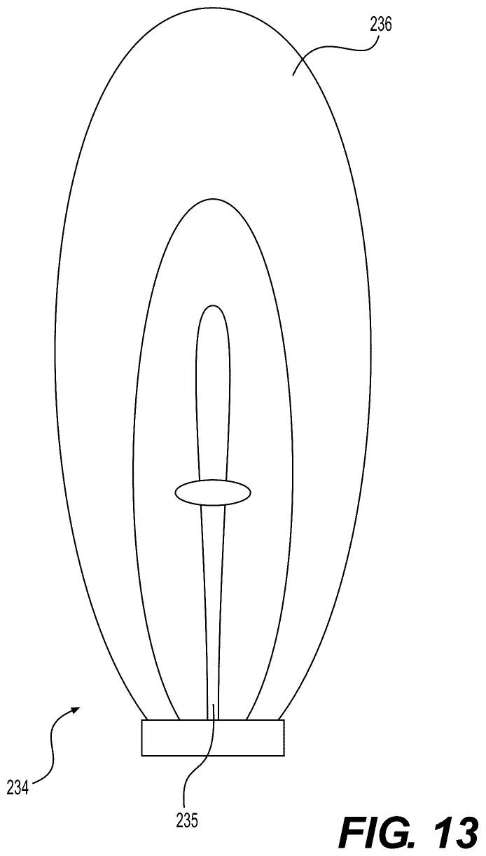

Each petal is formed of a soft, biocompatible material (e.g., silicone) having various different sections. For example, FIG. 13 shows a detailed view of a petal 234 of the speculum 200. The petal 234 has a generally oval shape. The petal 234 includes a rigid, elongated tine 235 centrally located on the petal 234 and extending from a proximal end towards a distal end of the petal 234. The elongated tine 235 is enclosed by a leaflet 236 that is softer than the elongated tine 235 (e.g., the leaflet 236 has a lower durometer than the tine 235). In some implementations, the petal may have a durometer that decreases from the tine to the radially outward edge of the petal such that the radially outward edge is the most flexible portion of the petal.

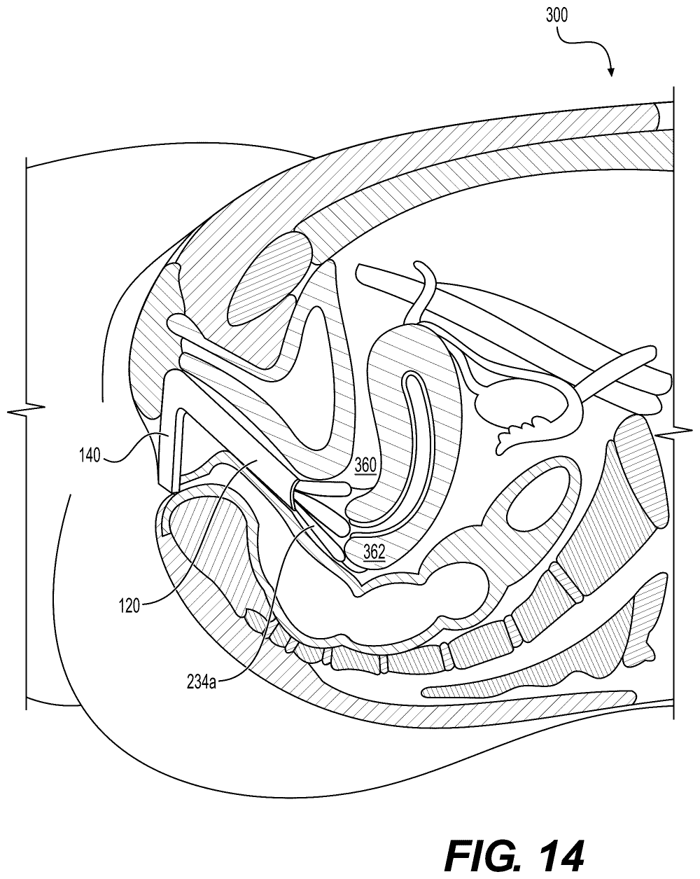

FIG. 14 shows a speculum 200 in use in a patient, wherein the expandable distal region 230 is in the open configuration, pushing aside the internal tissue 360 to better visualize cervix 362. The speculum 200 is operated and deployed similar to the speculum 100.

### EXAMPLE DEVICE #3

FIGS. 15 and 16 show an alternative deployment system 350 in a speculum 300. The expandable distal region 330 of the speculum 300 includes an outer portion 332 and a support portion 334 radially inward from the outer portion 332. The outer portion 332 is coupled to a first portion 322 of the proximal shaft 320. The support portion 334 is coupled to the outer portion 332 on one end and to a second portion 324 of the proximal shaft 320 on the other end.

The second portion 324 of the proximal shaft 320 is attached to a lead screw 352 of the deployment system 350. A shaft 354 extends from the lead screw 352 along the proximal shaft 320 and to a rotatable actuator 356 on a proximal end of the proximal shaft 320. In use, the rotatable actuator 356 is rotated to similarly rotate the shaft 354 and the lead screw 352.

Rotation of the lead screw 352 moves the support portion 334 of the expandable distal region 330 in a distal direction, which expands the outer portion 332 of the expandable distal region 330 radially outward. Thus, by rotating the rotatable actuator 356, the speculum 300 moves from the closed configuration to the open configuration.

In other implementations, the deployment system may be a spring, and the rotatable actuator instead pulls the spring back to expand the expandable distal region. In other implementations, the deployment system is a plunger/pusher style mechanism where the petals are initially compressed within the lumen of the proximal shaft and are pushed out and deployed.

### EXAMPLE SYSTEM AND METHOD

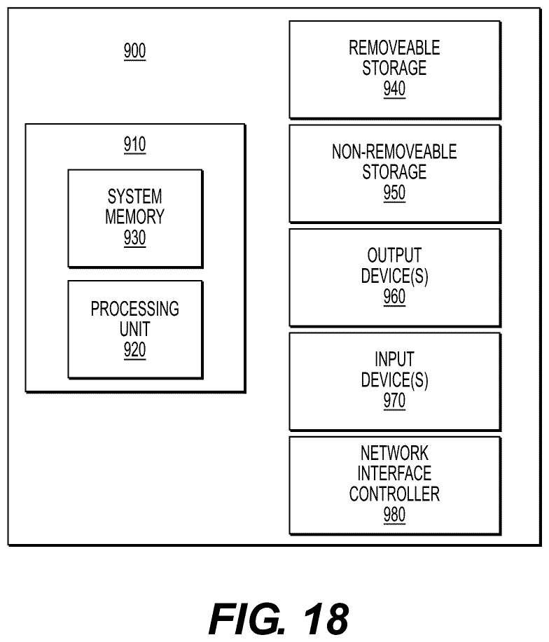

Further contemplated by this disclosure is a system for visualizing an internal anatomical structure. The system includes a speculum (e.g., the speculum 100 of FIG. 1) which includes an insertion portion (e.g., the insertion portion 110), a deployment mechanism (e.g., the deployment mechanism 150), and any other combination of speculum components described herein. The system further includes a controller (e.g., a controller 910 or computing device 900 as shown in FIG. 18).

In some implementations, the disclosed system and speculum device can visualize an anatomical structure in three dimensions (e.g., for vaginal or cervical assessment). For example, multiple angles of an endoscope (e.g., 30-degree angled insertion portions and/or endoscopes rotated to multiple different orientations), or multiple cameras of an endoscope, may be utilized to produce a three-dimensional model. Such three-dimensional models may be rendered for viewing on a separate screen or user interface, and they may be updated in relative real time.

The controller 910, further described below, may include a memory 930 configured to receive and store visual data from the endoscope of the speculum device. The controller 910 may further include a processor 920 configured to compile a plurality of images from the visual data to create a processed visual data set. The controller 910 may further include a user interface to display the processed visual data from the at least one camera of the endoscope.

The visual data captured by the endoscope may include a plurality of images taken from a plurality of angles. The visual data may also include a video or multiple videos taken from the endoscope.

Referring to FIG. 18, an example computing device 900 upon which embodiments of the invention may be implemented is illustrated. For example, the controller 910 of the speculum device and visualization system may be implemented as a computing device, such as computing device 900. It should be understood that the example computing device 900 is only one example of a suitable computing environment upon which embodiments of the invention may be implemented. Optionally, the computing device 900 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media. In an embodiment, the computing device 900 may comprise two or more computers in communication with each other that collaborate to perform a task.

Computing device 900 may have additional features/functionality. For example, computing device 900 may include additional storage such as removable storage 940 and non-removable storage 950 including, but not limited to, magnetic or optical disks or tapes. Computing device 900 may also contain network connection(s) 980 that allow the device to communicate with other devices such as over the communication pathways described herein. The network connection(s) 980 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 900 may also have input device(s) 970 such as a keyboards, keypads, switches, dials, mice, track balls, touch screens, voice recognizers, card readers, paper tape readers, or other well-known input devices. Output device(s) 960 such as a printers, video monitors, liquid crystal displays (LCDs), touch screen displays, displays, speakers, etc. may also be included. The additional devices may be connected to the bus in order to facilitate communication of data among the components of the computing device 900. All these devices are well known in the art and need not be discussed at length here.

The processing unit 920 may be configured to execute program code encoded in tangible, computer-readable media. Tangible, computer-readable media refers to any media that is capable of providing data that causes the computing device 900 (i.e., a machine) to operate in a particular fashion. Various computer-readable media may be utilized to provide instructions to the processing unit 920 for execution. Example tangible, computer-readable media may include, but is not limited to, volatile media, non-volatile media, removable media and non-removable media implemented in any method or technology for storage of information such as computer readable instructions, data structures, program modules or other data. System memory 930, removable storage 940, and non-removable storage 950 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 920 may execute program code stored in the system memory 930. For example, the bus may carry data to the system memory 930, from which the processing unit 920 receives and executes instructions. The data received by the system memory 930 may optionally be stored on the removable storage 940 or the non-removable storage 950 before or after execution by the processing unit 920.

Embodiments of the methods and systems may be described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

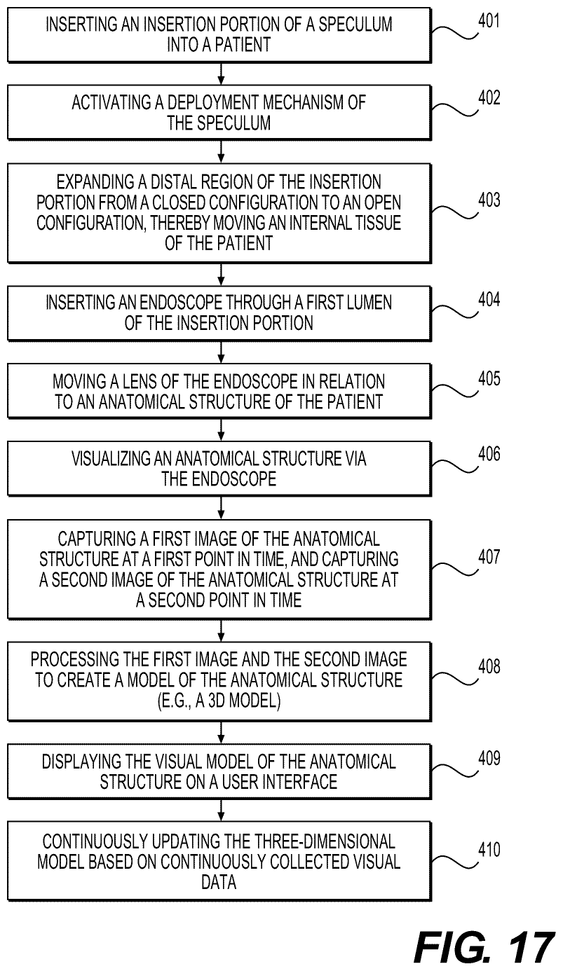

An example method of using the disclosed device and/or system is shown in FIG. 17. The method of FIG. 17 includes multiple steps provided in a certain order. However, in other implementations, any one of the steps of the disclosed methods may be replaced or removed depending on the application. In other implementations, any one of the steps of the disclosed method may be reordered in a different combination or permutation of the disclosed steps depending on the application.

At step 401, the method first includes inserting an insertion portion of a speculum (e.g., the speculum 100 of FIG. 1) into a patient. At step 402, the method includes activating a deployment mechanism of the speculum (e.g., to cause a fluid to flow along a lumen of the speculum). At step 403, the method includes expanding a distal region of the insertion portion from a closed configuration to an open configuration, thereby moving an internal tissue of the patient (e.g., for better visualization).

At step 404, the method includes inserting an endoscope through a first lumen of the insertion portion. At step 405, the method includes moving a lens or camera of the endoscope in relation to an anatomical structure of the patient. At step 406, the method includes visualizing an anatomical structure via the endoscope. At step 407, the method includes capturing a first image of the anatomical structure at a first point in time and capturing a second image of the anatomical structure at a second point in time (e.g., as in a video).

At step 408, the method includes processing the first image and the second image to create a model of the anatomical structure (e.g., a 3D model). At step 409, the method includes displaying the visual model of the anatomical structure on a user interface. At step 410, the method includes continuously updates the three-dimensional model based on continuously collected visual data.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps, and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

1. A speculum for improving visualization of an internal anatomical structure, the speculum comprising:
an insertion portion coupled to a handle, the insertion portion comprising a non-expandable proximal shaft and an expandable distal region, the distal region having a distal region length l that is less than 50% of a full length L of the insertion portion;
a deployment mechanism operably coupled to distal region;
wherein the proximal shaft comprises a first lumen configured for passage of an endoscope and a second lumen configured for passage of an instrument;
wherein the deployment mechanism controls movement of the expandable distal region between an open configuration and a closed configuration, wherein in the closed configuration a maximum width w of the distal region approximates a diameter D of the proximal shaft.

2. The speculum of claim 1, wherein the speculum further comprises a rotatable handle.

3-6. (canceled)

7. The speculum of claim 1, wherein further comprising a third lumen configured to be fluidically coupled to a contrast well for delivery of contrast agent.

8. The speculum of claim 1, wherein a starting point of the distal region is defined by a change in material from the proximal shaft.

9. (canceled)

10. The speculum of claim 1, wherein the distal region length l varies circumferentially.

11. The speculum of claim 1, wherein in the closed configuration, the maximum width w of the distal region is no more than 125% of the diameter D of the proximal shaft.

12. The speculum of claim 1, wherein in the open configuration, the distal region widens distally between a starting point and a distal edge region.

13. The speculum of claim 1, wherein in the open configuration, the distal region widens distally such that the maximum width w of the distal region is at least 250% of the diameter D of the proximal shaft.

14. The speculum of claim 13, wherein in the open configuration, the distal region widens in at least three circumferential directions.

15. The speculum of claim 14, wherein the distal region comprises at least three petals.

16. The speculum of claim 15, wherein at least one of the at least three petals comprises a rigid, elongated tine enclosed in a leaflet, the leaflet having a lower durometer than tine.

17. The speculum of claim 14, wherein the distal region comprises a funnel that widens in all circumferential directions.

18. The speculum of claim 17, wherein in the closed configuration, the funnel takes a retracted configuration, wherein the funnel is at least partially retracted within proximal shaft.

19. The speculum of claim 17, wherein in the closed configuration, the funnel takes a furled configuration.

20. The speculum of claim 1, wherein the deployment mechanism comprises an activator positioned on the handle.

21. (canceled)

22. The speculum of claim 20, wherein the deployment mechanism comprises an additional lumen extending at least partially between the activator and the distal region.

23. The speculum of claim 22, wherein the deployment mechanism comprises a fluid that travels through the additional lumen to fill the distal region in the open configuration.

24. The speculum of claim 22, wherein the deployment mechanism comprises a threaded portion and an internal rod extending at least partially between the activator and the threaded portion.

25-48. (canceled)

49. A system for visualizing an internal anatomical structure, the system comprising:
a speculum comprising:
an insertion portion coupled to a handle, the insertion portion comprising a non-expandable proximal shaft and an expandable distal region, the distal region having a distal region length l that is less than 50% of a full length L of the insertion portion;
a deployment mechanism operably coupled to distal region;
wherein the proximal shaft comprises a first lumen configured for passage of an endoscope comprising at least one camera on a distal end thereof and a second lumen configured for passage of an instrument;
wherein the deployment mechanism controls movement of the expandable distal region between an open configuration and a closed configuration, wherein in the closed configuration a maximum width w of the distal region approximates a diameter D of the proximal shaft; and
a controller in communication with the endoscope, the controller comprising:
a memory configured to receive and store visual data from the at least one camera of the endoscope; and
a processor configured to compile a plurality of images from the visual data to create a processed visual data set.

50. The system of claim 49, further comprising:
a user interface configured to display the processed visual data set from the at least one camera of the endoscope.

51-52. (canceled)
